(19) United States
(12) Patent Application Publication
(10) Pub. No.: US 2010/0062062 A1
(43) Pub. Date: Mar. 11, 2010

(54) Stabilized Coating for Pharmaceutical Formulations

(75) Inventors: Brian Robert MCMILLAN, Tampa, FL (US); Todd Roland DAVIAU, St. Petersburg, FL (US); John Michael CRONAN, JR., Brandon, FL (US); James Franklin DAVIS, III, Tarpon Springs, FL (US); Mark James LICARDE, St. Petersburg, FL (US); Saurabh Sudhir TRIVEDI, Riverview, FL (US); Ian W. COTTRELL, Spring Hill, FL (US)

Correspondence Address: SALIWANCHIK LLOYD & SALIWANCHIK;A PROFESSIONAL ASSOCIATION, PO Box 142950, GAINESVILLE, FL, 32614, US

(73) Assignee: AETHOS PHARMACEUTICALS, INC., Tampa, FL (US)

(21) Appl. No.: 12/556,839

(22) Filed: Sep. 10, 2009

Related U.S. Application Data
(60) Provisional application No. 61/096,124, filed on Sep. 11, 2008.

(51) Int. Cl. A61K 9/32; A61K 9/16; A61K 31/40

(52) U.S. Cl. 424/465; 424/490; 514/412

(57) ABSTRACT

A process is described for preparing stabilized tablet formulations for temperature and moisture sensitive active drugs. Water soluble polyvinyl alcohol is processed with drugs such as angiotensin converting enzyme (ACE) inhibitors and compressed into solid form once excess water is removed. Low dose polyvinyl alcohol ramipril tablets prepared by this process are stable under conditions of high humidity and heat for periods of at least up to six months with less than 8% hydrolysis of the prodrug to the active metabolite diketopiperazine (DKP).

This application takes priority from U.S. Provisional Application Ser. No. 61/096,124 filed Sep. 11, 2008, the contents of which are incorporated by reference.

### BACKGROUND OF THE INVENTION

1. Field of the Invention

The invention relates to drug coatings, particularly polymer based coatings conferring long range shelf life and thermal stability.

2. Description of Background Art

Discovery, development and marketing a drug is a time consuming and expensive process. Once safety and efficacy have been established, appropriate formulations may be different for each class of drug and even for drugs with similar structure and activity. A significant amount of testing is typically involved to determine the best mode of administration, whether by injection, topical administration or oral route, including whether or not an oral formulation is therapeutically effective.

For most drugs, oral formulations are preferable, and are typically supplied as solids in the form of tablets or powders encased in gelatin capsules. Unfortunately, for some drugs, safety and efficacy, while acceptable in fresh preparations, are not maintained during storage or distribution. Shelf life is an important consideration in maintaining inventory availability and quality of the product.

A major concern of drug manufacturers is long-term stability of solid pharmaceutical agents, not only with respect to loss of activity but also for some classes of drugs, the potential for degradation products to produce toxins or compounds that can lead to unwanted side effects. Many approaches to increasing shelf life and protecting against thermal degradation rely on various coating methods to protect active compounds from humidity and atmospheric oxygen.

Solid drug forms are generally preferable for manufacture and formulation since tablets and capsule forms are among the most popular of dispensed medications. Several classes of therapeutic agents have stability issues, leading to the need to display dated shelf life, special sealed bottles, or light-tight and refrigerated storage conditions. These may include but are not limited to ACE inhibitors, anti-convulsants, anti-hypertensive, alzheimers drugs, anti-depressants, anti-psychotics, psychotherapeutics, diuretics, agents for treating irritable bowel syndrome, anti-hyper lipidemic, osteo-regulatory, thrombolytics and vasodilators. These classes include widely used drugs that typically require additional cost in manufacturing and storage in order to meet safety and efficacy requirements.

ACE inhibitors are among the most important classes of drugs used in the treatment of essential hypertension and heart failure. These drugs include ramipril, benazepril, captopril, enalapril, lisinopril, fosinopril, perindopril, quinapril, moexipril and trandolapril. One of the most popular and most frequently prescribed is ramipril, sold as Altace® capsules. Unfortunately, ramipril is susceptible to degradation caused by the mechanical compression required to manufacture tablets, and also exhibits a strong sensitivity to heat and moisture. Other members of this class such as enalapril also exhibit instability under normal storage conditions.

Ramipril is not the active form of the drug. In the presence of air and moisture, it undergoes cyclization via internal nucleophilic attack to form a diketopiperazine (DKP), which is the active form of the drug. Any excessive amount of DKP can be highly detrimental when given to a patient because the prodrug metabolizes at a calculated rate to the proper therapeutic amount. Any excess DKP originally present in the formulation may lead to overdosing with subsequent kidney and/or liver damage.

There are numerous reports addressing the instability of ramipril which is generally ascribed to a combination of such factors as heat, moisture, oxidation and in the case of tablets, the compression processes used in manufacture. Efforts to stabilize ACE inhibitor drugs have focused on protective coatings, and the use of select binders, and/or additives.

U.S. Pat. No. 7,160,558 describes a coating/binding agent based on a composition made from an acrylic or methacrylic acid copolymer, an emulsifier and a monocarboxylic acid. The polymer coating and binding film was moisture resistant and when sprayed on placebo tablets appeared to provide initial protection from moisture when the coated tablets were stored in environmentally controlled cabinets at 40° C. and 75% humidity. After 24 hr. however, water absorption was up to half the amount absorbed by uncoated tablets after 24 hr.

Polymer coatings are also described in U.S. Pat. No. 4,705,695 which claims a method for coating solid pharmaceutical agents with acrylic and/or methacrylic based polymers having tertiary amine side groups and optionally mixed with various additional agents such as talcum powder, lubricant or polyethylene glycol. Chlorpheniramine maleate pellets were spray coated with the polymer and tested for dissolution in water or synthetic digestive juice. Release of the drug was about 80% after 6 hours while other tests in water showed rapid dissolution as quickly as 2 minutes after exposure to an aqueous medium.

U.S. Pat. No. 7,175,857 describes a granulate or powder formulated from an acrylic or methacrylic based polymer which can be dissolved and mixed with a pharmaceutically active compound to form a coating on the compound after the mixture is cooled. The resulting granulated powders had a specified water vapor permeability. Quinidine sulfate tablets were spray coated with the described polymer and said to form a uniform coating on the tablet with imperviousness to taste over a period of at least 30 minutes.

Efforts to effect stabilization of active drugs that exhibit decomposition or destabilization when compressed into tablets have been reported in U.S. Pat. No. 5,151,433 and the related U.S. Pat. No. 5,442,008. ACE inhibitors coated with polymeric film formers were found to significantly reduce the effect of the compression applied during the tablet forming process, which appeared to be the main cause of drug destabilization in the ACE inhibitor ramipril. A number of polymers were listed as possibly suitable for protective films, including several cellulose and polymethacrylate based polymers, polyvinyl acetate phthalate, and polyvinylpyrrolidone. The examples utilized hydroxypropylmethylcellulose (HPMC) as the polymer. When coated with HPMC, ramipril tablets stored for 12 months at 40° C. in tight screw glass containers showed about 24% of the DKP measured in the dry ramipril which had not been compressed into tablets.

### SUMMARY OF THE INVENTION

The present invention is based on use of a polymer coating on moisture and temperature sensitive pharmaceutically active agents. The polymer is coated on individual particles and provides high stability for compressed formulations and powders. In contrast to many conventional polymers employed to stabilize drugs, it has been found that polyvinyl alcohol will efficiently coat and stabilize drugs in particulate or powder form without resorting to the relatively complex or expensive processes currently used.

ACE inhibitors and drugs such as those listed above are among the classes of compounds notably sensitive to heat and/or moisture. Ramipril, sold as Altace®, is particularly challenging because it tends to rapidly hydrolyze to diketopiperazine, DKP, which is the active drug form. When in the form of tablets, storage conditions cannot be entirely controlled so that moisture and heat can promote degradation and formation of high levels of DKP. Initially ingesting undetermined amounts of DKP in combination with the normal in vivo formation of DKP can lead to overdosing of the active form of the drug.

Decomposition of low dose ramipril, 1.25 mg for example, under normal manufacturing and storage conditions has effectively prevented companies from producing low dose solid forms of this class of drugs. The use of polyvinyl alcohol as a stabilizing coating for ramipril tablets would not initially appear to offer any advantages over other polymers that might be contemplated for coating drug particles. Polyvinyl acetate phthalate, for example, while water soluble and therefore convenient for efficiently mixing with and coating finely dispersed or dissolved solids, has to be heated in order to remove water. The heating causes decomposition of the polyvinyl acetate phthalate to phthalic acid, which is an unacceptable impurity. Thus there was no reason to believe based on polymer coating results reported by others nor was there any indication in the art to particularly contemplate that polyvinyl alcohol would provide any particular advantage over other polymers in providing a stabilizing coating. In fact the conventional methods for coating involving spray drying failed to provide a protective coating on solid ramipril (typically used as API) when polyvinyl alcohol was randomly tested as a possible polymer stabilizing coating.

Despite poor results with spray dry conventional methods for polymer coating of solids, it was found that polyvinyl alcohol mixed under high shear granulation conditions with uncoated ramipril active pharmaceutical ingredient (API) provides stabilized material that can be dried, mixed with desired additives and compressed into tablets without decomposition. The tablets are not only initially stable to compression, but also exhibit long-term storage stability to heat and humidity. A shelf life of at least 6 months is a distinct commercial advantage and is especially important when drugs must be shipped or stored in locations without climate control. Moreover, in contrast to other reports using polymer coatings for stabilization, low dose tablets, e.g., 1.25 mg ramipril, when prepared using the described polyvinyl alcohol coating process, are quite stable to heat and moisture. This was a distinct advantage because the process of mixing, high shearing of the solid and freeze drying or using a fluid bed granulator/dryer allowed drying without heat decomposition. Thus the simple step of high shear processing with polyvinyl alcohol provided a simple effective process for obtaining a stable coating for heat/moisture sensitive drugs such as ramipril formulated in low dose tablet form.

The coating process is preferably a high shear granulation process in which a heat/moisture sensitive drug is coated with a polyvinyl alcohol solution about 0.1% to about 20% by weight, preferably 2,5, 3.0, 3.5, 4.0, 4.5 or 5% which creates a coating level ranging from 0.1% to 20%, depending on the initial polyvinyl alcohol concentration, on individual drug particles. Alternatively, the coated drug is processed and dried by conventional means before addition of selected additives and compression into tablets.

While the invention is particularly directed toward the stabilization of ACE inhibitor drugs, other classes of drugs such as anti-convulsants, anti-hypertensive, anti-depressants, anti-psychotics, psychotherapeutics, diuretics, anti-hyper lipidemics, osteo-regulatory, thrombolytics and vasodilators often exhibit sensitivity to compression processes and to heat and moisture, particularly in low dosage formulations. Stabilization of compressed forms of these drugs by way of processing the active ingredient with polyvinyl alcohol and selected excipients such as release modifiers, disintegrants, bulking agents, lubrication agents, stability agents and the like, can lead to the formation of a coated pharmaceutically active agent once excess water is removed. Other water soluble alcohol similar to polyvinyl alcohol may also be suitable coating agents when used on highly granulated therapeutically active solids in the described process for tablets.

The ACE inhibitor ramipril tends to be unstable in pharmaceutical formulations depending on contact with excipients in the manufacturing process as well as storage conditions of either capsules or tablets. Inhibiting decomposition of the prodrug ramipril to diketopeperazine product (DKP) is important because this compound is the active form of ramipril, which is metabolized in the body to DKP. In addition, ramipril solid also decomposes on exposure to air, heat and/or moisture to a diacid, ramiprilat, which is undesirable because it effectively lowers the drug dose.

Oral forms of drugs absorbed in the small intestine may be degraded in the stomach and therefore not taken into the body. Additional stabilization of tablet forms of ramipril and other drugs sensitive to acid decomposition may be achieved by coating granulated drug with a fat or wax, either simultaneously or step-wise with polyvinyl alcohol. This is expected to provide added stability toward gastrointestinal absorption so that low dose formulations are more effective. This also would address individual differences in patients with different gastric acidity. Lipid materials have been used in this manner in formulations of anti-parasitic compounds. Examples of lipids suitable for co-coating are found in Application Serial No. 2006/0068020 and Application Serial No. 2006/0067954. In choosing a lipid such as a fat or wax as a coating with polyvinyl alcohol, biocompatibility is a factor as is solubilization. For example, long chain fatty acids can be dissolved in alcohol with polyvinyl alcohol and then used to coat the highly granulated drug before drying and compressing.

### BRIEF DESCRIPTION OF THE DRAWING

FIG. 1 is a graph showing 6-month stability of 1.25 mg ramipril tablets stored at 40° C. at 75% humidity. Two batches are shown, each having less than 8% DPK at the end of the storage period.

### DETAILED DESCRIPTION OF THE INVENTION

The present invention provides a process for producing a stabilized compressed formulation for active agents that are susceptible to decomposition when exposed to moisture and heat. The described process utilizes a water soluble polymer, namely polyvinyl alcohol, admixed with the highly pulverized therapeutic agent and selected optional additives to prepare a coated stable solid after excess water and/or alcohol are removed. The polymer coated particulates can be compressed into tablets or other compressed forms and still remain stable to heat, moisture and air.

Tablet forms of the ACE inhibitor ramipril are well recognized in the art and commercially as particularly susceptible to degradation and are notably unstable in low dose formulations such as 1.25 mg. As discussed, ramipril itself is a prodrug that converts in the body to the active form, diketopiperazine via a cyclization reaction, and to a lesser extent to ramiprilat, a diacid metabolite arising from hepatic cleavage of the ramipril ester group. Polyvinyl coated ramipril particles in compressed tablet form have a shelf life of at least 6 months, and are stable to heat and moisture. DKP formation even after 6 months is less than 8%, the maximum amount acceptable in ramipril tablets as specified in the British Pharmacopeia. Tablet dosage formulations in the range of 1-20 mg with polyvinyl alcohol coatings as described herein meet all current standards for purity, specifically decomposition to the active drug form, DKP.

There are several methods for processing polyvinyl alcohol and ramipril to provide stabilized polymer coated tablets. An important aspect of coating ramipril, or other heat/moisture sensitive solid drug, is to use a method that finely pulverizes or fluidizes polyvinyl alcohol and the drug. At least two effective methods are illustrated; one by pulverizing ramipril by some means such as mortar and or mechanical rotary blade mixer with a low percent of polyvinyl alcohol, e.g., 3.5-5%, in water or absolute ethanol; and another by spraying a solution of polyvinyl alcohol onto a complex of ramipril API and PROSOLV using a 15-90% drug load in a high shear granulator or fluid bed granulator/dryer.

When using the pulverization method, the aqueous or alcoholic mixture of polyvinyl alcohol and ramipril can be freeze dried, typically at −25° C. When dried, the material is screened to a mesh size of about 20 or desired particulate size before mixing with any of a number of additives, as desired. This is preferably accomplished using a blender such as a v-shell PK Blend Master Blender. Additives may include PROSOLV SMCC90®, sodium starch glycolote, and sodium stearyl fumarate as desired. At this stage, tablets can be obtained using a rotary tablet press or other compression means suitable for tableting.

In a second process for making stabilized ramipril tablets, polyvinyl alcohol solution is sprayed onto a complex of ramipril API and PROSOLV SMCC® utilizing for example a 20% drug load in a fluid bed granulator/drying. The complex can be dried in the fluid bed granulator/dryer to less than 4% moisture. Once solid material is obtained, it can be screened through a screen of desired mesh, typically 20 mesh. The meshed complex is diluted with PROSOLV SMCC90®, sodium starch glycolote and sodium stearyl fumarate and blended. Once blended the material can be compressed into tablets using known manufacturing processes.

Alternatively, aqueous polyvinyl alcohol can be sprayed onto a complex of ramipril API and PROSOLV SMCC® and granulated in a high shear granulator and dried in a fluid bed granulator/dryer as described. To add additional stability to ramipril after oral administration, a solution of polyvinyl alcohol and an alcohol soluble wax or fat can be sprayed onto the complex before granulating and drying. Several biocompatible lipids may be suitable and are described in U.S. Application Serial Number 2006/0068020, incorporated herein by reference with respect to exemplary lipids, including but not limited to long chain fatty acids such as palmitic or oleic acid.

Storage tests at different temperatures and relative humidity were conducted on the tablets to assess resistance to degradation under relatively long storage times. Degradation to DKP was measured over a period of several months and provided data showing a high resistance to DKP formation under relative humidity of up to 75% and at temperatures up to 40° C. when stored in HDPE bottles with a moisture scavenger like desiccant or molecular sieve and induction sealed caps on the bottles.

### Examples

The following examples are provided as illustrations of the invention and are in no way to be considered limiting.

Materials

Ramipril was purchased from Trademax Pharmaceuticals and Chemical Co., LTD, 100% API, batch #20070302, 98% min purity. Polyvinyl alcohol, 87-89% partially hydrolyzed was obtained from J. T. Baker, U232-08, Lot E26585. Ethanol was from Acros, 99.5%, ACS reagent grade and water used was USP grade. Prosolv is a mixture of colloidal silica (2%) and microcrystalline cellulose (98%).

Silicified microcrystalline cellulose; colloidal silicon dioxide, Vivastar-starch glycolate, NF grade, and Pruv-sodium steryl fumarate were from JRS Pharma. PROSOLVE® and SMCC90®.

The following examples illustrate the stabilizing coating process used for preparing ramipril tablets. The amount of ramipril active pharmaceutical ingredient (API) typically was 1.25, 2.5, 5, 10 or 20 mg.

### Example 1

### Polyvinyl Alcohol Coated Ramipril

Uncoated ramipril (Trademax Pharmaceuticals and Chemical Co, LTD, (Shanghai, China) was granulated in a mortar and pestle or with a kitchen type blender and mixed with a 3-5% partially hydrolyzed polyvinyl alcohol solution in ethanol, 60% ethanol or purified water. The blended material was frozen to −80° C. and freeze dried for 24 hr. The resulting solid was screened through a 20-mesh screen.

The coated ramipril was mixed with silicified microcrystalline cellulose and colloidal silicon dioxide and sodium stearyl fumarate using a v-shell blender and then compressed into tablets on a rotary tablet press.

Tablet hardness, thickness and weight were measured on selected batch samples. Typical hardness was in the range of 8-10 kp and friability less than 0.1%. Tablet diameter was ¼ in and weight 100 mg.

The tablets were placed in 60 cc high density polyethylene (HDPE) white round bottles containing a moisture scavenger desiccant (about 1 g) or molecular sieve. Bottle caps were inductively sealed and stored at 25° C., 60% relative humidity and 40° C. and 75% relative humidity. Bottles were randomly selected at different times and the 1.25 mg ramipril tablets were tested for DKP using liquid chromatography. Analyses were compared against standards for ramipril and DKP.

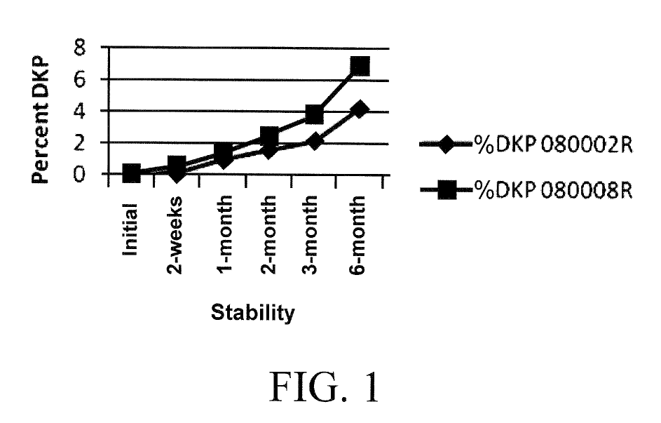

FIG. 1 shows typical stability results for 1.25 mg ramipril tablets over a 6-month period stored at 40° C. and 75% relative humidity. At the end of 6 months the amount of DKP was less than the 8% limit imposed by the British Pharmacopia.

Table 1 shows 6-month stability tests for low dose ramipril tablets prepared with polyvinyl alcohol coatings and for ramipril tablets stored coated with hydroxypropylmethylcellulose and stored under the conditions reported in U.S. Pat. No. 5,442,008 (the '008 patent).

TABLE 1

| Storage | | | | |
|---|---|---|---|---|
| (months) | Temperature | Rel. humidity (%) | DKP (%) | Amt (mg) |
| 6 (example 1) | 40° C. | 75 | 4.2 | 1.25 |
| 6 (′008 patent)¹ | 40° C. | Airtight | 1.87 | 2.5 |

¹Data taken from U.S. Pat. No. 5,442,008

### Example 2

### Polyvinyl Alcohol Coated Ramipril

A 10% (w/w) polyvinyl solution in water was sprayed onto a mixture of PROSOLV, silicified microcrystalline cellulose and ramipril at 20% load in a fluid bed granulator/dryer. Diluents such as microcrystalline cellulose, lactose, starch and the like can optionally be added to the initial mixture. The mixture was dried in the granulator/dryer at about 50° C. inlet temperature for a time sufficient to produce a solid suitable for screening through a 20-mesh screen.

The coated screened ramipril material was diluted with silicified microcrystalline cellulose, colloidal silicon dioxide, starch glycolate and sodium stearyl fumarate using a v-shell blender and then compressed on a rotary tablet press. Tablet hardness, thickness, friability and weight were recorded. Friability was less than 0.5%.

The tablets were then stored in HDPE bottles containing a moisture scavenger desiccant or molecular sieve. Bottle caps were inductively sealed and stored at 25° C., 60% relative humidity and 40° C., 75% relative humidity. Bottles were randomly selected at different times and the tablets were tested for DKP using liquid chromatography. Analyses were compared against a ramipril and a DKP standard.

### Example 3

### Polyvinyl Alcohol Coated Ramipril

Polyvinyl alcohol in purified water (5% w/w) was sprayed onto a mixture of ramipril and silicified microcrystalline cellulose (PROSOLV SMCC) using 80% drug load granulated in a high shear granulator. The mixture was then dried in a fluid bed granulator/dryer to less than 4% moisture before screening through a 20-mesh screen.

The screened material was mixed with PROSOLV SMCC90®, sodium starch glycolate and stearyl fumarate in a v-shell blender, removed and compressed into tablets on a rotary tablet press.

The tablets were then stored in HDPE bottles containing a moisture scavenger desiccant or molecular sieve. Bottle caps were inductively sealed and stored at 25° C.60% relative humidity and 40° C., 75% relative humidity. Bottles were randomly selected at different times and the tablets tested for DKP using liquid chromatography. Analyses were compared against a ramipril and a DKP standard.

### Example 4

### Polyvinyl Alcohol Coated Ramipril

Six month stability tests under 75% relative humidity at 40° C. were conducted on tablets prepared as described in the above examples and stored for 6 months. Stability was indicated by the amount of DKP formed as shown in Table 2. Although DKP was below the accepted 8% limit according to the British Pharmacopia, polyvinyl alcohol in the range of 3-5% provided more stabilization than 10% polyvinyl alcohol.

TABLE 2

| DKP (w/w) | 40° C., 75% relative humidity |
|---|---|
| 3.0% | 3.5% polyvinyl alcohol in ethanol granulation |
| 3.6% | 5% polyvinyl alcohol in water granulation |
| 6.9% | 10% polyvinyl alcohol in water granulation |
| 4.2% | 5% polyvinyl alcohol high sheer granulation |

### Example 5

### Low Dose 1.25 mg Ramipril Tablets

Co-sprayed ramipril with PROSOLV was compared with uncoated ramipril. The 1.25 mg ramipril vinyl alcohol coated tablets were assayed for ramipril and decomposition products DKP and ramiprilat. The results are shown in Table 3. Similar results were obtained using 1%-8% polyvinyl alcohol coatings. Typical coatings were 2% on tablets with 80% drug.

TABLE 3

| | | | Area | Area % |
|---|---|---|---|---|
| Sample I.D. | Description | Assay % L.C. | % DKP | Ramiprilat |
| RD001F001A | Co-Sprayed | 54.3 | 38.0 | 1.7 |
| | Ramipril with | | | |
| | PROSOLV | | | |
| RD002F002A | Control Batch | 96.8 | 17.8 | 0.55 |
| | with Uncoated | | | |
| | Ramipril | | | |
| 1087156-TS | Co-Sprayed | 70.9 | 32.5 | 1.3 |
| | Ramipril with | | | |
| | PROSOLV | | | |

### Example 6

### Vinyl Alcohol/Lipid coatings for Gastrointestinal Stability

Additional stability of the described coated tablets after oral ingestion can be achieved by co-coating granulated ramipril with both polyvinyl alcohol and a high melting wax or fat. This is expected to provide additional stability in gastrointestinal absorption. A selected wax or fat is liquified by heating to the appropriate temperature, which will be below the decomposition temperature of the active ingredients that are to be mixed with the liquefied wax or fat. The source of lipids can consist of a single component “hard butter”, which refers to a lipid system that has characteristics and/or a solid fat melting index similar to cocoa butter and is similar in rapid meltdown characteristics. Exemplary lipids may include partially hydrogenated vegetable oil, soybean oil, cottonseed oil, palm oil and a mixture of palm oil and palm kernel oil. The lipid system could also consist of petroleum wax, vegetable or animal stearates, a high sharp melting point vegetable fat, or combinations of hard butters and stearates. It is also possible to use mineral oil or petrolatum. The lipid base should have a melting point of about 80 to 130° F.

When using a co-coating of lipid or wax for granulated ramipril, the selected lipid or wax is preferably dissolved in alcohol or similar solvent to admix with the drug and vinyl alcohol in order to avoid heat degradation. Once the highly granulated drug, vinyl alcohol and lipid are mixed, the coated ramipril tablets are prepared as described in the examples herein.

### REFERENCES

U.S. Pat. No. 5,442,008 (Fulberth, et al.)

U.S. Pat. No. 5,151,433 (Fulberth, et al.)

U.S. Pat. No. 7,175,857 (Petereit, et al.)

U.S. Pat. No. 7,160,558 (Petereit, et al.)

U.S. Application Serial No. 2008/0234353

U.S. Application Serial No. 20060177498

U.S. Application Serial No. 2006/0068020

U.S. Application Serial No. 2006/0067954

What is claimed is:

1. A stabilized compressed pharmaceutical formulation, comprising an admixture of a highly granulated pharmaceutical agent coated with polyvinyl alcohol wherein the polyvinyl alcohol provides a long-term stabilized moisture and temperature protective coating on the granulated pharmaceutical agent.

2. The formulation of claim 1 wherein the admixture further comprises a binder or excipient.

3. The formulation of claim 2 wherein the excipient is selected from cellulose, microcrystalline cellulose, silicified-microcrystalline cellulose, sodium starch glycolate, sodium stearyl fumarate, lactose and dicalcium phosphate.

4. The formulation of claim 2 wherein the admixture further comprises an additive for lubrication, melting, casting, spreading, spraying or granulating.

5. The formulation of claim 1 wherein the pharmaceutical agent is an angiotensin converting enzyme (ACE) inhibitor, anti-convulsant, anti-hypertensive, anti-depressant, anti-psychotic, psychotherapeutic, diuretic, anti-hyper lipidemic, osteo-regulatory, thrombolytic or vasodilator.

6. The formulation of claim 5 wherein the drug class is an ACE inhibitor.

7. The formulation of claim 6 wherein the ACE inhibitor is selected from the group consisting of ramapril, benazepril, captopril, enalapril, lisinopril, fosinopril, perindopril, quinapril, moexipril and trandolapril.

8. The formulation of claim 7 wherein the ACE inhibitor is ramipril.

9. The formulation of claim 1 wherein the compressed form is a tablet.

10. The compressed formulation of claim 1 further comprising a biocompatible wax or lipid coating on the granulated pharmaceutical agent, wherein the wax or lipid is mixed with the polyvinyl alcohol coating.

11. A 1-25 mg ramipril tablet comprising polyvinyl alcohol coated ramipril particles.

12. The tablet of claim 11 which comprises 1.25 mg ramipril.

13. The tablet of claim 11 further comprising one or more suitable binders or pharmaceutically acceptable excipients.

14. Polyvinyl alcohol coated ramipril particulates comprised within 1.0 to 2.5 mg ramipril tablets which degrade to less than 8% diketopiperazine (DKP) during storage time of at least 12 months in containers exposed to relative humidity up to at least 75%.

15. The coated ramipril tablets of claim 14 wherein the storage temperature is up to 40° C.

16. A process for the manufacture of the compressed pharmaceutical formulation of claim 1 comprising mixing about 0.1% to about 20% polyvinyl alcohol aqueous or ethanolic solution with the highly granulated pharmaceutical agent of claim 1 or spraying a complex comprising the pharmaceutical agent and microcrystalline cellulose with 5% to about 10% aqueous polyvinyl alcohol to form a complex, and optionally adding one or more pharmaceutically acceptable tablet diluents, drying the complex, screening the dried complex, and compressing said dried complex into compressed form.

17. The process of claim 16 wherein the highly granulated pharmaceutical agent has a particulate size less than about 20-mesh or 840 micron.

18. The process of claim 16 wherein the drying is by freeze drying or fluid bed granulator/dryer.

19. The process of claim 16 wherein the pharmaceutical agent is ramipril.

20. The process of claim 19 wherein the ramipril is 1 to about 25 mg in the compressed formulation.

21. The process of claim 16 wherein the compressed formulation is a tablet comprising 1.25 mg ramipril.

22. The process of claim 21 wherein the ramipril tablet comprises granulated ramipril coated with a mixture of vinyl alcohol and a stabilizing biocompatible wax or long-chain fatty acid.
